(19) United States
(12) Patent Application Publication
(10) Pub. No.: US 2021/0330261 A1
(43) Pub. Date: Oct. 28, 2021

(54) SENSOR ARRAY AND DEVICE

(71) Applicant: Samsung Electronics Co., Ltd., Suwon-si (KR)

(72) Inventors: Jiyoung JUNG, Seoul (KR); Youngjun YUN, Seongnam-si (KR); Suk Gyu HAHM, Gyungju-si (KR)

(73) Assignee: Samsung Electronics Co., Ltd., Suwon-si (KR)

(21) Appl. No.: 17/094,082

(22) Filed: Nov. 10, 2020

(30) Foreign Application Priority Data
Apr. 23, 2020 (KR) 10-2020-0049549

(51) Int. Cl. A61B 5/00; H01L 31/173; H01L 31/0392; A61B 5/024; A61B 5/0488

(52) U.S. Cl. CPC A61B 5/6833; H01L 31/173; H01L 2031/0344; A61B 5/02416; A61B 5/0488; H01L 31/03926

(57) ABSTRACT

Disclosed are a sensor array and a device including the sensor. In the sensor array in which a plurality of unit element groups are arranged, each of the unit element groups includes a pressure sensor, a light emitting element, and/or a light detecting element, and the pressure sensor includes a variable resistance layer including a stretchable polymer and conductive nanostructures dispersed in the stretchable polymer.

### CROSS-REFERENCE TO RELATED APPLICATION

This application claims priority to and the benefit of Korean Patent Application No. 10-2020-0049549 filed in the Korean Intellectual Property Office on Apr. 23, 2020, the entire contents of which are incorporated herein by reference.

### BACKGROUND

### 1. Field

A sensor array and a device are disclosed.

### 2. Description of the Related Art

Recently, research on attachable devices (wearable devices) that directly attach biological devices such as smart skin devices, soft robots, and biomedical devices to skin or clothing has been conducted.

### SUMMARY

Some example embodiments provide a newly structured sensor array applicable to an attached device.

Some example embodiments provide a device including the sensor array.

According to some example embodiments, a sensor array is provided. In the sensor array in which a plurality of unit element group are arranged, each of the unit element group includes a pressure sensor, a light emitting element, and/or a light detecting element, and the pressure sensor includes a variable resistance layer including a stretchable polymer and conductive nanostructures dispersed in the stretchable polymer.

The stretchable polymer may include a polyorganosiloxane, an elastomer including a butadiene moiety, an elastomer including a urethane moiety, an elastomer including an acrylic moiety, an elastomer including an olefin moiety, an inorganic elastomer, a derivative thereof, or a combination thereof.

The conductive nanostructures may include carbon nanotubes, carbon nanowires, carbon nanoplates, carbon nanoflakes, carbon nanofibers, carbon nanocomposites, carbon nanoparticles, metal nanotubes, metal nanowires, metal nanoplates, metal nanoflakes, metal nanofibers, metal nanocomposites, metal nanoparticles, graphene, or a combination thereof.

An aspect ratio of the conductive nanostructures may be greater than or equal to about 10.

The conductive nanostructures may be included in an amount of about 0.001 wt % to about 50 wt % based on a total amount of the stretchable polymer and the conductive nanostructures.

The pressure sensor may further include a pair of electrodes.

A resistance change rate of the pressure sensor in the pressure range of about 15 kPa to 35 kPa may be greater than or equal to about 20%.

The light emitting element may include a first light emitting element and a second light emitting element which emit light in different wavelength spectra from each other.

The first light emitting element may be a red light emitting element configured to emit light in a red wavelength spectrum, and the second light emitting element may be a green light emitting element configured to emit light in a green wavelength spectrum.

The light emitting element may include an inorganic light emitting diode, an organic light emitting diode, a micro light emitting diode, or a combination thereof.

The light emitting element may be a stretchable light emitting element.

The light detecting element may include an inorganic photodiode, an organic photoelectric conversion element, or a combination thereof.

The light detecting element may be a stretchable light detecting element.

Each unit element group may include one pressure sensor, two light emitting elements, and one light detecting element.

According to some example embodiments, a device including the sensor array is provided.

The device may further include a stretchable substrate supporting the sensor array.

The device may be a skin-attachable patch type or a skin-attachable band type.

The device may be a photoplethysmography sensor device, an electromyography sensor device, or a strain sensor device.

According to some example embodiments, a method of operating the sensor array is provided. The method includes specifying a position of a pressure sensor in which a pressure is sensed among a plurality of unit element group, and selectively driving unit element group including the pressure sensor in which the pressure is sensed.

The selectively driving of the unit element group including the pressure sensor configured to sense the pressure may include irradiating first light from the light emitting element of the unit element group including the pressure sensor sense the pressure, and absorbing second light generated by reflection of the first light by an object by the light detecting element to convert the absorbed second light into an electrical signal.

Sensitivity may be improved by increasing variation of resistance according to the pressure of the pressure sensor, and thus, pressure may be stably sensed. In addition, by sensing a position where a predetermined or alternatively, desired pressure occurs in the sensor array, a predetermined or alternatively, desired unit element group is selectively driven to increase sensing sensitivity and accuracy.

### BRIEF DESCRIPTION OF THE DRAWINGS

FIG. 1 is a schematic view showing an example of a pixel array of a sensor array according to some example embodiments,

FIG. 2 is a schematic view showing a portion of an example of the sensor array of FIG. 1,

FIG. 3 is a cross-sectional view showing an example of a pressure sensor in the sensor array of FIGS. 1 and 2,

FIGS. 4A and 4B are cross-sectional views showing an example of the variable resistance layer in the pressure sensor of FIG. 3,

FIGS. 5 and 6 are schematic views showing application examples of the device according to some example embodiments,

FIG. 7 is a schematic view showing an example of an operation of a biosensor device according to some example embodiments, and

FIG. 8 is a graph showing a resistance variation rate depending on a pressure of the pressure sensors according to example and comparative example.

### DETAILED DESCRIPTION

Hereinafter, example embodiments will be described in detail so that those skilled in the art can easily implement them. However, the actual applied structure may be implemented in various different forms and is not limited to the implementations described herein.

In the drawings, the thickness of layers, films, panels, regions, etc., are exaggerated for clarity. It will be understood that when an element such as a layer, film, region, or substrate is referred to as being “on” another element, it may be directly on the other element or intervening elements may also be present. In contrast, when an element is referred to as being “directly on” another element, there are no intervening elements present.

Hereinafter, the term “combination” includes a mixture and a stacked structure of two or more.

When the term “about” is used in this specification in connection with a numerical value, it is intended that the associated numerical value include a tolerance of ±10% around the stated numerical value. When ranges are specified, the range includes all values therebetween such as increments of 0.1%.

Hereinafter, a sensor array according to some example embodiments is described with reference to the drawings.

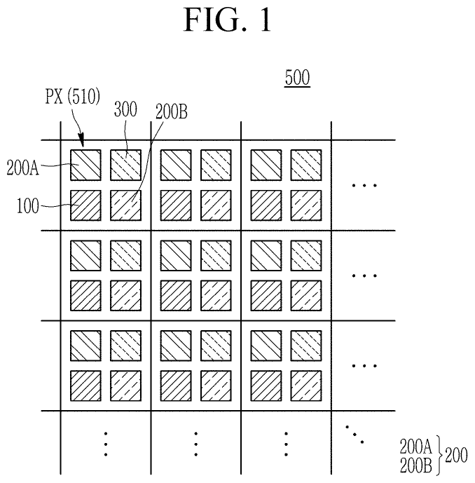

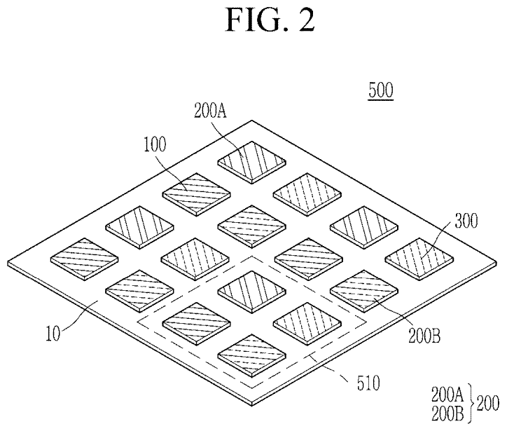

FIG. 1 is a schematic view showing an example of a pixel array of a sensor array according to some example embodiments, and FIG. 2 is a schematic view showing a portion of an example of the sensor array of FIG. 1.

Referring to FIG. 1, the sensor array 500 includes a plurality of pixels PXs, and the plurality of pixels PXs may have a matrix arrangement repeatedly arranged along rows and/or columns. An arrangement of the pixels PXs may be, for example, a Bayer matrix, a PenTile matrix, and/or a diamond matrix, but is not limited thereto.

Although all the pixels PXs are shown to have the same size in the drawing, the present disclosure is not limited thereto, and one or more pixels PXs may be larger or smaller than other pixels PXs. Although all pixels PXs are illustrated in the drawing as having the same shapes, the present disclosure is not limited thereto, and one or more pixels PXs may have shapes different from those of the other pixels PXs.

For example, each pixel PX may include a unit element group 510.

Referring to FIG. 2, each unit element group 510 may be arranged on the substrate 10. The substrate 10 may be a stretchable substrate, and thus may flexibly respond to external forces or external movements such as twisting, pressing, and pulling, and may be easily restored to its original state.

The substrate 10 may include, for example, an elastomer. The elastomer may include an organic elastomer, an organic/inorganic elastomer, an inorganic elastomer-like material, or a combination thereof. The organic elastomer or organic/inorganic elastomer may include a substituted or unsubstituted polyorganosiloxane such as polydimethylsiloxane; an elastomer including a substituted or unsubstituted butadiene moiety such as styrene-ethylene-butylene-styrene; an elastomer including a urethane moiety; an elastomer including an acrylic moiety; an elastomer including an olefin moiety; or a combination thereof, but is not limited thereto. The inorganic elastomer-like material may include, elastic ceramics, elastic solid metals, liquid metals, or a combination thereof, but is not limited thereto.

Each unit element group 510 may be driven independently, and may include switching and/or driving elements, such as thin film transistors for switching and/or driving for each pixel PX.

Each unit element group 510 includes a pressure sensor 100, a light emitting element 200, and/or a light detection element 300. The pressure sensor 100, the light emitting element 200, and/or the light detecting element 300 included in each unit element group 510 may each independently be one or two or more.

The pressure sensor 100, the light emitting element 200, and/or the light detecting element 300 included in each unit element group 510 may have a dimension of several to several tens of micrometers. For example, the pressure sensor 100, the light emitting element 200, and/or the light detecting element 300 included in each unit element group 510 may independently have each width, length, and thickness of greater than or equal to about 0.1 μm and less than about 100 μm, for example each width, length, and thickness of about 0.1 μm to about 80 μm, about 0.1 μm to about 70 μm, about 0.1 μm to about 60 μm, about 0.1 μm to about 50 μm, about 0.1 μm to about 40 μm, about 0.1 μm to about 30 μm, about 0.1 μm to about 20 μm, about 0.1 μm to about 10 μm, or about 0.1 μm to about 5 μm.

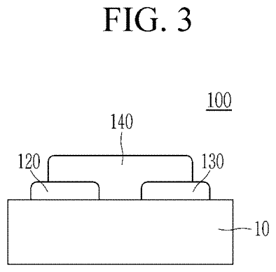

FIG. 3 is a cross-sectional view showing an example of a pressure sensor 100 in the sensor array of FIGS. 1 and 2, and FIG. 4 is a cross-sectional view showing an example of the variable resistance layer 140 in the pressure sensor 100 of FIG. 3.

The pressure sensor 100 is a sensor configured to sense pressure applied from the outside.

Referring to FIG. 3, the pressure sensor 100 in each unit element group 510 includes a pair of electrodes 120 and 130 and a variable resistance layer 140 which are disposed on the substrate 10.

The pair of electrodes 120 and 130 may be made of, for example, a metal, a conductive oxide, a conductive polymer, or a combination thereof, for example, gold (Au), copper (Cu), nickel (Ni), aluminum (Al), molybdenum (Mo), chromium (Cr), tantalum (Ta), titanium (Ti), an alloy thereof, zinc oxide, indium oxide, tin oxide, indium tin oxide (ITO), indium zinc oxide (IZO), or fluorine-doped tin oxide, poly(3,4-ethylenedioxythiophene) (PEDOT), poly(3,4-ethylenedioxythiophene) polystyrene sulfonate (PEDOT:PSS), or a combination thereof, but are not limited thereto.

At least one of the pair of electrodes 120 and 130 may be a stretchable electrode. For example, the stretchable electrode may include a stretchable conductor, or may have a stretchable shape such as a wavy shape, a wrinkle shape, a popup shape, or a non-coplanar mesh shape.

The variable resistance layer 140 may change a resistance according to a pressure applied from the outside. For example, when no pressure is applied, the variable resistance layer 140 is substantially insulated and thus exhibits a high resistance value, whereas when a predetermined or alternatively, desired pressure is applied, the variable resistance layer 140 may exhibit a reduced resistance value. According to the change of the resistance value, it is possible to detect whether a pressure is generated or not or to sense a pressure intensity.

The variable resistance layer 140 may have a relatively low stiffness. Herein, the stiffness indicates a degree of resistance to deformation when a force is applied from the outside, and a relatively low stiffness means that the resistance to deformation is relatively small, so that the deformation is large. The stiffness can be evaluated from an elastic modulus and the elastic modulus may be, for example, Young's modulus. The elastic modulus of the variable resistance layer 140 may be, for example, about 10² Pa to about 10⁷ Pa, but is not limited thereto.

The variable resistance layer 140 may have a relatively high elongation rate. Here, the elongation rate may be a percentage of a length change that is increased to a breaking point relative an initial length. For example, the elongation rate of the variable resistance layer 140 may be greater than or equal to about 10%, and within the range, about 10% to about 500%, about 10% to about 400%, about 10% to about 300%, about 10% to about 200%, about 10% to about 100%, about 10% to about 90%, about 10% to about 80%, about 10% to about 70%, about 10% to about 60%, about 10% to about 50%, about 10% to about 40%, about 20% to about 70%, about 20% to about 60%, about 20% to about 50%, or about 20% to about 40%.

The variable resistance layer 140 may flexibly respond to external forces or external movements such as twisting and pulling due to the relatively low stiffness and relatively high elongation rate, and may be easily restored to its original state.

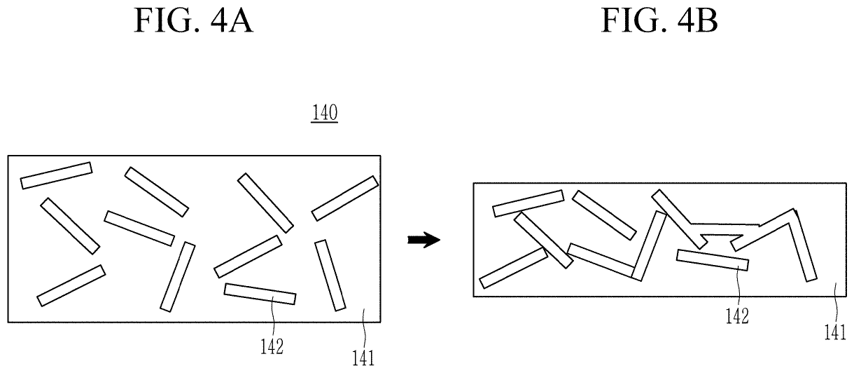

Referring to FIG. 4, the variable resistance layer 140 includes a stretchable polymer 141 and a plurality of conductive nanostructures 142.

The stretchable polymer 141 may include a polymer having stretchability, for example, an organic or organic/inorganic elastomer of polyorganosiloxane such as polydimethylsiloxane (PDMS), an elastomer including a butadiene moiety such as styrene-ethylene-butylene-styrene (SEBS), an elastomer including a urethane moiety, an elastomer including an acrylic moiety, an elastomer including an olefin moiety, a derivative thereof, or a combination thereof, but is not limited thereto.

The conductive nanostructures 142 may be, for example, a structure having a dimension of several to several tens of nanometers, and may include, for example, nanotubes, nanowires, nanoplates, nanoflakes, nanofibers, nanocomposites, nanoparticles, or a combination thereof, but are not limited thereto. For example, the conductive nanostructure 142 may include carbon nanotubes, carbon nanowires, carbon nanoplates, carbon nanoflakes, carbon nanofibers, carbon nanocomposites, carbon nanoparticles, metal nanotubes, metal nanowires, metal nanoplates, metal nanoflakes, metal nanofibers, metal nanocomposites, metal nanoparticles, graphene, or a combination thereof, but is not limited thereto.

For example, the conductive nanostructures 142 may be wire-type nanostructures that are long in one direction, and may have an aspect ratio of greater than or equal to about 10. For example, the conductive nanostructures 142 may be carbon nanotubes, carbon nanowires, carbon nanoplates, carbon nanoflakes, carbon nanofibers, carbon nanocomposites, metal nanotubes, metal nanowires, metal nanoplates, metal nanoflakes, metal nanofibers, metal nanocomposites, or a combination thereof which have an aspect ratio of about 10 to about 10^{6,} about 10² to about 10^{6,} or about 10³ to about 10^{6,} but are not limited thereto.

The plurality of conductive nanostructures 142 are dispersed in the stretchable polymer 141. That is, the stretchable polymer 141 may serve as a matrix of the variable resistance layer 140. For example, the plurality of conductive nanostructures 142 are substantially uniformly dispersed in the stretchable polymer 141, and the spacing between adjacent conductive nanostructures 142 may be sufficiently maintained.

For example, referring to FIGS. 4A and 4B, when no pressure is applied (4A), the conductive nanostructures 142 may maintain a sufficient distance from each other and substantially maintain an insulating state. On the other hand, when a predetermined or alternatively, desired pressure is applied (4B), it may be easily deformed due to the relatively low stiffness and relatively high elongation rate of the variable resistance layer 140, and thus, adjacent conductive nanostructures 142 are brought to be close or be contact with each other and an insulation state may be broken. That is, due to the relatively low stiffness and relatively high elongation rate of the variable resistance layer 140, a resistance value of the variable resistance layer 140 may be sensitively changed depending on the presence or absence of pressure, and the resistance may be greatly reduced when it is changed from a state without a pressure to a state with a predetermined or alternatively, desired pressure. From this change in resistance, it is possible to detect whether pressure is presented or not and a pressure intensity.

The change in the resistance may be adjusted according to the content of the conductive nanostructures 142, and the content of the conductive nanostructures 142 may be adjusted according to required sensitivity of the pressure sensor 100. For example, the conductive nanostructure 142 may be included in an amount of greater than or equal to about 0.0001 wt % relative to a total amount of the stretchable polymer 141 and the conductive nanostructures 142, and within the range, may be included in an amount of about 0.0001 wt % to about 50 wt %, about 0.001 wt % to about 50 wt %, about 0.01 wt % to about 50 wt %, about 0.0001 wt % to about 40 wt %, about 0.001 wt % to about 40 wt %, about 0.01 wt % to about 40 wt %, about 0.0001 wt % to about 30 wt %, about 0.001 wt % to about 30 wt %, about 0.01 wt % to about 30 wt %, about 0.0001 wt % to about 20 wt %, about 0.001 wt % to about 20 wt %, about 0.01 wt % to about 20 wt %, about 0.0001 wt % to about 10 wt %, about 0.001 wt % to about 10 wt %, about 0.01 wt % to about 10 wt %, about 0.0001 wt % to about 5 wt %, about 0.001 wt % to about 5 wt %, or about 0.01 wt % to about 5 wt %.

For example, the resistance variation rate of the pressure sensor at a pressure range of about 15 kPa to about 35 kPa may be greater than or equal to about 20%, and within the range, may be greater than or equal to about 30%, greater than or equal to about 40%, greater than or equal to about 50%, greater than or equal to about 60%, greater than or equal to about 70%, greater than or equal to about 80%, about 20% to about 100%, about 30% to about 100%, about 40% to about 100%, about 50% to about 100%, about 60% to about 100%, about 70% to about 100%, or about 80% to about 100%. Here, the resistance variation rate may be expressed as (R−R₀)/R₀×100, wherein R may be a resistance value when pressure is applied, and R₀ may be an initial resistance value when no pressure is applied.

Accordingly, as described above, in the sensor array 500 in which the plurality of unit element groups 510 is arranged in a matrix format, when a pressure is applied to a part of pixels (PX), a position where a pressure is applied may be specified by using a resistance change of the pressure sensor 100 in the corresponding pixel (PX), and accordingly, the unit element group 510 of the corresponding pixel (PX) alone may be selectively operated and thus effectively detect the specific position of an object (not shown).

The light emitting element 200 included in each unit element group 510 may be configured to emit light in a predetermined or alternatively, desired wavelength spectrum, and may include, for example, an inorganic light emitting diode, an organic light emitting diode, or a micro light emitting diode. The light emitting element 200 may include, for example, a pair of electrodes and a light emitting layer between the pair of electrodes. For example, the pair of electrodes may be stretchable electrodes, the stretchable electrodes may, for example, include a stretchable conductor or have a stretchable shape such as a wavy shape, a wrinkle shape, a popup shape, or a non-coplanar mesh shape. For example, the light emitting layer may include an organic light emitting material, a quantum dot, and/or perovskite, but is not limited thereto. For example, the pair of electrodes may be stretchable electrodes, and the light emitting layer may be a stretchable light emitting layer, and accordingly, the light emitting element 200 may be, for example, a stretchable element.

For example, each unit element group 510 may include a plurality of light emitting elements 200 configured to emit light of different wavelength spectra from each other, for example, the light emitting elements 200 may include one or two or more selected from a red light emitting element configured to emit light in a red wavelength spectrum, a green light emitting element configured to emit light in a green wavelength spectrum, a blue light emitting element configured to emit light in a blue wavelength spectrum, an infrared light emitting element configured to emit light in an infrared wavelength spectrum, an ultraviolet (UV) light emitting element configured to emit light in an ultraviolet (UV) wavelength spectrum, or a combination thereof.

For example, as shown in FIGS. 1 and 2, each unit element group 510 may include two light emitting elements 200 configured to emit light in two different wavelength spectra from each other, and these two light emitting elements 200 configured to emit light in the different wavelength spectra from each other may be used to detect objects having different absorption and/or reflection characteristics. For example, the light emitting element 200 may include a red light emitting element 200A configured to emit light in a red wavelength spectrum and a green light emitting element 200B configured to emit light in a green wavelength spectrum, and the red light emitting element 200A and the green light emitting element 200B may be used for absorption and/or reflection characteristics of oxyhemoglobin HbO₂ and hemoglobin Hb in a blood vessel.

The light detecting element 300 included in each unit element group 510 may be configured to absorb light, for example, include an inorganic photodiode, an organic photoelectric conversion element, or a combination thereof. The light detecting element 300 may include, for example, a photoelectric conversion layer between the pair of electrodes. For example, the pair of electrodes may be stretchable electrodes, and the stretchable electrodes may, for example, include a stretchable conductor or have a stretchable shape such as a wavy shape, a wrinkle shape, a popup shape, or a non-coplanar mesh shape. For example, the photoelectric conversion layer may include, for example, an inorganic semiconductor, an organic semiconductor, and/or an organic/inorganic semiconductor, for example, a p-type semiconductor and an n-type semiconductor forming a pn junction. For example, the photoelectric conversion layer may be a stretchable photoelectric conversion layer. The light detecting element 300 may be, for example, a stretchable light detecting element.

The sensor array 500 may further include an encapsulation film (not shown) covering the aforementioned pressure sensor 100, light emitting element 200, and light detecting element 300. The encapsulation film may be isolated respectively on the pressure sensor 100, the light emitting element 200, and/or the light detecting element 300 or be entirely disposed on the pressure sensor 100, the light emitting element 200, and/or the light detecting element 300.

The encapsulation film may include, for example, organic materials, inorganic materials, and/or organic materials, and may include one or more layers. For example, the encapsulation film may include an inorganic material such as oxide, nitride, and/or oxynitride, and/or an organic material such as a photosensitive elastomer. For example, the encapsulation film may include oxide, nitride, and/or oxynitride including at least one of aluminum (Al), titanium (Ti), zirconium (Zr), hafnium (Hf), tantalum (Ta) and silicon (Si); a substituted or unsubstituted polysiloxane such as polydimethylsiloxane; a photosensitive elastomer in which a substituted or unsubstituted an elastomer including a substituted or unsubstituted butadiene moiety such as styrene-ethylene-butylene-styrene; an elastomer including a urethane moiety; an elastomer including an acrylic moiety; an elastomer including an olefin moiety, or a combination thereof. For example, the elastomer may include the above moiety as a main chain of the elastomer and further include a photosensitive functional group bonded to the main chain, such as a substituted or unsubstituted vinyl group or a substituted or unsubstituted (meth)acrylic group.

For example, the encapsulation film is formed by alternately stacking layers having different refractive indexes, for example, a first layer including a first material selected from oxide, nitride, and oxynitride, and a second layer including a second material selected from oxide, nitride, and oxynitride and having a higher refractive index than that of the first layer.

The encapsulation film may protect the sensor array 500 including the unit element groups 510 and effectively block, reduce or prevent inflow of oxygen, moisture and/or contaminants from the outside. For example, when the sensor array 500 is included in a wearable device attached to a living body, the encapsulation film may reduce or prevent inflow of biological secretions such as sweats into the sensor array 500 and thus degradation of the sensor array 500.

As described above, the unit element group 510 of each pixel PX includes the pressure sensor 100, the light emitting element 200, and/or the light detecting element 300, and the pressure sensor 100 is used to specify a position where a predetermined or alternatively, desired pressure is applied and thus operate only the unit element group 510 at the position of the sensor array and resultantly, increase accuracy of a sensor.

This sensor array 500 may be applied to various devices requiring pressure sensing and stretchability, for example, a skin-like device, a large-area conformable display, smart clothing, and the like, but is not limited thereto.

For example, the aforementioned sensor array 500 may be applied to an attachable biosensor device. The attachable biosensor device may further include an integrated circuit (IC) (not shown) and/or a processor (not shown) for processing information obtained from the aforementioned sensor array 500. The attachable biosensor device may further include a display element (not shown). The display element may display the obtained information in the aforementioned sensor array 500 into predetermined or alternatively, desired information such as various letters and/or images.

The attachable biosensor device may be a very thin patch-type or band-type and attached to the surface of a living body such as a skin, inside the living body such as an organ, or indirect means of contact with the living body such as clothing and thus sense and measure bio-information such as bio-signals. For example, the attachable biosensor device may be a skin-attachable patch type or a skin-attachable band type and attached to a living body and thus monitor bio-information in real time.

For example, the attachable biosensor device may be an electroencephalogram (EEG) sensor device, an electrocardiogram (ECG) sensor device, a blood pressure (BP) sensor device, an electromyography (EMG) sensor device, a blood glucose (BG) sensor device, a photoplethysmography (PPG) sensor device, an accelerometer device, a RFID antenna device, an inertial sensor device, an activity sensor device, a strain sensor device, a motion sensor device, or a combination thereof, but is not limited thereto.

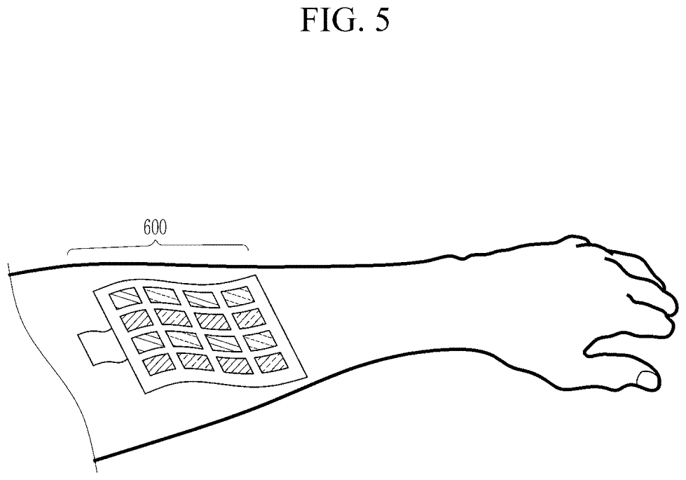

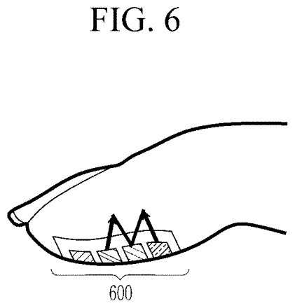

FIGS. 5 and 6 are schematic views each showing an example of applying an attachable biosensor device to a living body.

Referring to FIGS. 5 and 6, the attachable biosensor device 600 may be a photoplethysmography (PPG) sensor device and attached on arms or fingers and thus sense bio-information such as a blood pressure, a pulse, oxygen saturation, arrhythmia, and stress, and the bio-information may be obtained by analyzing a waveform of electrical signals.

For another example, the biosensor device 600 may be an electromyography (EMG) sensor device or a strain sensor device attachable to joints for rehabilitation of patients having joint and muscle problems, and the bio-information may include, for example, muscle motions, joint motions, or the like and be obtained by analyzing a waveform of electrical signals. This bio-information may be quantitatively measured to secure data required for the rehabilitation.

For example, a method of operating the biosensor device may include specifying a position of the pressure sensor 100 which detects a pressure among the plurality of unit element group 510 of the sensor array 500, and selectively driving the unit element group 510 to which the pressure sensor 100 detecting the pressure belongs. The selectively driving of the unit element groups 510 to which the pressure sensor 100 configured to sense the pressure belongs may, for example, include irradiating light from the light emitting element 200 of the unit element groups 510 to which the pressure sensor 100 belongs, and absorbing light reflected by an object such as a blood vessel by the light detecting element 300 and converting the absorbed light into electrical signals.

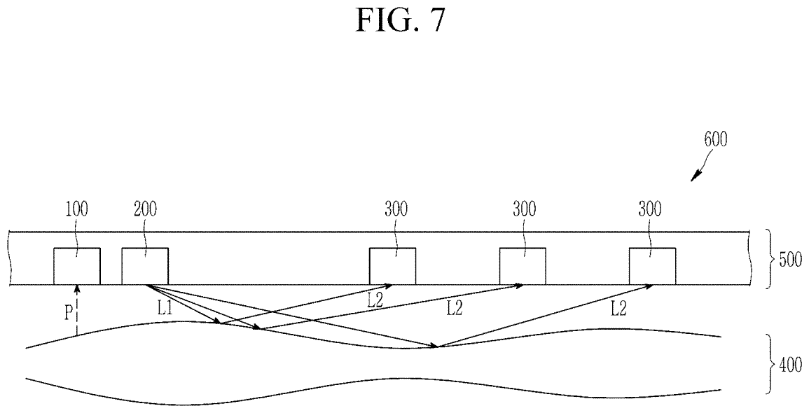

FIG. 7 is a schematic view showing an example of an operation of a biosensor device.

Referring to FIG. 7, the biosensor device 600 includes the aforementioned sensor array 500 including the pressure sensor 100, the light emitting element 200, and/or the light detecting element 300. When the sensor array 500 is attached to a living body, a pressure may be detected by a resistance change of the pressure sensor 100 where a predetermined or alternatively, desired pressure (P) such as a blood pressure occurs, and the light emitting element 200 of the unit element group 510 to which the corresponding pressure sensor 100 belongs may emit light (L1) to blood vessel 400 for sensing bio-signals. The light (L1) may be reflected by a part of the living body (e.g., skin, blood vessel, or muscle) (400), and the reflected light (L2) may be absorbed by the light detecting element 200 and converted the absorbed light into the electrical signals. Herein, the plurality of light detecting elements 200 adjacently positioned one another may obtain different values depending on a distance and treat the electrical signals in various methods to increase accuracy of the sensor. The electrical signals converted from the reflected light (L2) may include bio-information. The electrical signals including the bio-information may be transferred to a sensor IC (not shown) or a processor (not shown).

Hereinafter, example embodiments are illustrated in more detail with reference to examples. However, these examples are exemplary, and the present scope is not limited thereto.

### Example

Au is deposited on a styrene-ethylene-butylene-styrene (SEBS) substrate to form a source electrode and a drain electrode. Subsequently, a mixed solution of carbon nanotube dispersion dispersed in toluene at a concentration of 0.1 wt % per 1 ml of toluene and styrene-ethylene-butylene-styrene (SEBS, concentration: 100 mg/1 ml) is twice to three times dropped between the source electrode and the drain electrode, and dried in a vacuum oven or on a hot plate at 80° C. for 1 hour to form a variable resistance layer and thus manufacturing a pressure sensor.

### Comparative Example

Au is deposited on a SEBS substrate to form a source electrode and a drain electrode. Subsequently, carbon nanotube dispersion dispersed in water at a concentration of 0.1 wt % is twice to three times dropped between the source electrode and the drain electrode and dried in a vacuum oven or on a hot plate at 80° C. for 1 hour to form a variable resistance layer and thus manufacturing a pressure sensor.

### Evaluation

Resistance variations depending on the pressure of the pressure sensor according to Example and Comparative Example are evaluated.

The resistance variations are calculated from current changes of the variable resistance layers, and resistance variation rates are calculated according to (R−R₀)/R₀×100 (R: resistance, R₀: initial resistance).

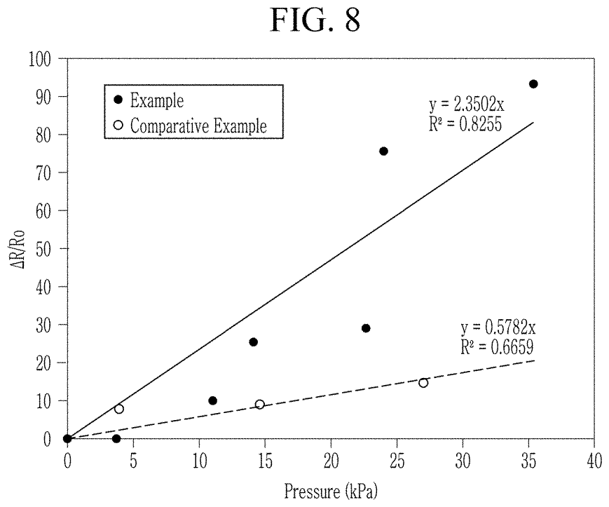

The results are shown in FIG. 8 and Table 1.

FIG. 8 is a graph showing a resistance variation rate depending on a pressure of the pressure sensors according to example and comparative example.

TABLE 1

| | Sensitivity |
|---|---|
| Example | 3.14 |
| Comparative Example | 0.38 |

Referring to FIG. 8 and Table 1, the pressure sensor according to Example exhibits large resistance variation depending on a pressure change compared with the pressure sensor according to Comparative example and thus may realize a pressure sensor having higher sensitivity.

While this disclosure has been described in connection with what is presently considered to be practical example embodiments, it is to be understood that the inventive concepts are not limited to the disclosed example embodiments. On the contrary, it is intended to cover various modifications and equivalent arrangements included within the spirit and scope of the appended claims.

What is claimed is:

1. A sensor array, the sensor array comprising a plurality of unit element groups,
wherein each of the unit element groups comprises a pressure sensor, a light emitting element, and a light detecting element, and
the pressure sensor comprises a variable resistance layer comprising a stretchable polymer and conductive nanostructures dispersed in the stretchable polymer.

2. The sensor array of claim 1, wherein the stretchable polymer comprises a polyorganosiloxane, an elastomer comprising a butadiene moiety, an elastomer comprising a urethane moiety, an elastomer comprising an acrylic moiety, an elastomer comprising an olefin moiety, an inorganic elastomer, a derivative thereof, or a combination thereof.

3. The sensor array of claim 1, wherein the conductive nanostructures comprise carbon nanotubes, carbon nanowires, carbon nanoplates, carbon nanoflakes, carbon nanofibers, carbon nanocomposites, carbon nanoparticles, metal nanotubes, metal nanowires, metal nanoplates, metal nanoflakes, metal nanofibers, metal nanocomposites, metal nanoparticles, graphene, or a combination thereof.

4. The sensor array of claim 3, wherein an aspect ratio of the conductive nanostructures be greater than or equal to about 10.

5. The sensor array of claim 1, wherein the conductive nanostructures are included in an amount of about 0.001 wt % to about 50 wt % based on a total amount of the stretchable polymer and the conductive nanostructures.

6. The sensor array of claim 1, wherein the pressure sensor further comprises a pair of electrodes.

7. The sensor array of claim 1, wherein a resistance variation rate of the pressure sensor in a pressure range of about 15 kPa to 35 kPa is greater than or equal to about 20%.

8. The sensor array of claim 1, wherein the light emitting element comprises a first light emitting element and a second light emitting element which is configured to emit light in different wavelength spectra from each other.

9. The sensor array of claim 8, wherein
the first light emitting element is a red light emitting element configured to emit light in a red wavelength spectrum, and
the second light emitting element is a green light emitting element configured to emit light in a green wavelength spectrum.

10. The sensor array of claim 1, wherein the light emitting element comprises an inorganic light emitting diode, an organic light emitting diode, a micro light emitting diode, or a combination thereof.

11. The sensor array of claim 10, wherein the light emitting element is a stretchable light emitting element.

12. The sensor array of claim 1, wherein the light detecting element comprises an inorganic photodiode, an organic photoelectric conversion element, or a combination thereof.

13. The sensor array of claim 12, wherein the light detecting element is a stretchable light detecting element.

14. The sensor array of claim 1, wherein each unit element group comprises one pressure sensor, two light emitting elements, and one light detecting element.

15. The sensor array of claim 1, further comprising a stretchable substrate supporting the pressure sensor, the light emitting element and the light detecting element.

16. A device comprising the sensor array of claim 1.

17. The device of claim 16, wherein the device is a skin-attachable patch type device or a skin-attachable band type device.

18. The device of claim 16, wherein the device is a photoplethysmography sensor device, an electromyography sensor device, or a strain sensor device.

19. A method of operating the sensor array of claim 1, comprising
specifying a position of a pressure sensor where a pressure is sensed among a plurality of unit element groups, and
selectively driving unit element groups comprising the pressure sensor in which the pressure is sensed.

20. The method of claim 19, wherein
the selectively driving of the unit element groups comprising the pressure sensor configured to sense the pressure comprises:
irradiating first light from the light emitting elements of the unit element groups comprising the pressure sensors configured to sense the pressure, and
absorbing second light generated by reflection of the first light by an object by the light detecting elements to convert the absorbed second light into an electrical signal.
